(19) United States
(12) Patent Application Publication
(10) Pub. No.: US 2001/0047027 A1
(43) Pub. Date: Nov. 29, 2001

(54) Prostaglandin D2 receptor antagonists

(76) Inventors: Marc Labelle, Burlingame, CA (US); Claudio Sturino, Dorval (CA); Roy Bruno, Ile Bizard (CA)

Correspondence Address: MERCK AND CO INC, P O BOX 2000, RAHWAY, NJ, 070650907

(21) Appl. No.: 09/817,987

(22) Filed: Mar. 27, 2001

Related U.S. Application Data
(60) Provisional application No. 60/196,316, filed on Apr. 12, 2000.

(51) Int. Cl. A61K 31/403; C07D 209/82

(52) U.S. Cl. 514/411; 548/439

(57) ABSTRACT

Certain tetrahydrocarbazole-1-acetic acid derivatives are potent and selective antagonists of the prostaglandin D2 receptor, and are therefore useful in the treatment of allergic conditions such as allergic rhinitis.

### BACKGROUND OF THE INVENTION

Histamine, cysteinyl leukotrienes (CysLTs), prostaglandin D2 (PGD2) and thromboxane A₂ (TxA₂) are considered to be key mediators in allergic conditions such as allergic rhinitis, allergic asthma and allergic conjunctivitis (Chan et al., 1989; Narita et al., 1996; Yamasaki et al., 1997; Yasui et al., 1997; Fujita et al., 1997). Released by activated mast cells they have been shown to increase microvascular permeability, blood flow, intranasal pressure and mucus secretion. These mediators assert their physiological effects primarily through interaction with their respective receptors; accordingly, treatments for allergic conditions have included agents that can block or otherwise interrupt such interactions. For example, anti-histamines and leukotriene D4 receptor antagonists have been shown previously to be effective in a guinea pig model of allergic rhinitis and conjunctivitis. (Chan et al., 1989). Leukotriene antagonists are now part of the arsenal for the treatment of asthma, and antihistamines have long been used to treat symptoms of allergic rhinitis. Because allergic conditions are attributed to multiple mediators, blocking the interaction of one mediator with its receptor may not be sufficient to alleviate the symptoms often associated with allergic conditions.

While antihistamines have been shown efficacious for preventing and relieving sneezing, itching, rhinorrhea and other symptoms of the early allergic response, they have not been found to be very effective for relief of the nasal blockage which is characteristic of the later stages of an allergic reaction. Prostaglandin D₂ (PGD₂) is also thought to be involved in human allergic rhinitis, a frequent allergic disease that is characterized by itch, sneezing, rhinorrhea and nasal congestion (Baraniuk, 1998; Doyle et al., 1990; Raphael et al., 1991; Ramis et al., 1991). Nasal provocation with PGD₂ provoked a dose-dependent increase in nasal congestion, the most manifest symptom of allergic rhinitis (Doyle et al., 1990). In addition, elevated levels of PGD₂ were noted in the nasal wash fluid of allergic patients that underwent a nasal antigen challenge.

U.S. Pat. No. 4,808,608 discloses tetrahydrocarbazole-1-alkanoic acids as prostaglandin antagonists. The compound 9-p-chlorobenzyl-8-methylsulfinyl-2,3,4,9-tetrahydro-1H-carbazol-1-yl-acetic acid (1), as a diastereomeric mixture, is specificially disclosed therein.

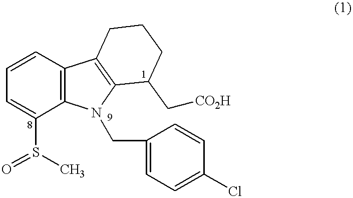

Applicants have now discovered that the stereoconfiguration at position 1 of the tetrahydrocarbazole ring is determinative of the compound's affinity for the prostaglandin D2 receptor. The 1R isomers have much higher affinity for the receptor than the 1S isomers.

### SUMMARY OF INVENTION

The present invention provides 2-[(1R)-9-(4-chlorobenzyl)-8-methylsulfinyl-2,3,4,9-tetrahydro-1H-carbazol-1-yl]acetic acid, pharmaceutical compositions thereof, and methods for their use in the treatment of allergic conditions.

### DETAILED DESCRIPTION OF THE INVENTION

The present invention provides substantially pure 2-[(1R)-9-(4-chloro-benzyl)-8-methylsulfinyl-2,3,4,9-tetrahydro-1H-carbazol-1-yl]acetic acid (I) consisting essentially of 2-[(1R)-9-(4-chlorobenzyl)-8-((S)-methylsulfinyl)-2,3,4,9-tetrahydro-1H-carbazol-1-yl]acetic acid and 2-[(1R)-9-(4-chlorobenzyl)-8-((R)-methylsulfinyl)-2,3,4,9-tetrahydro-1H-carbazol-1-yl]acetic acid of formula I(a) and I(b), respectively, and pharmaceutically acceptable salts thereof.

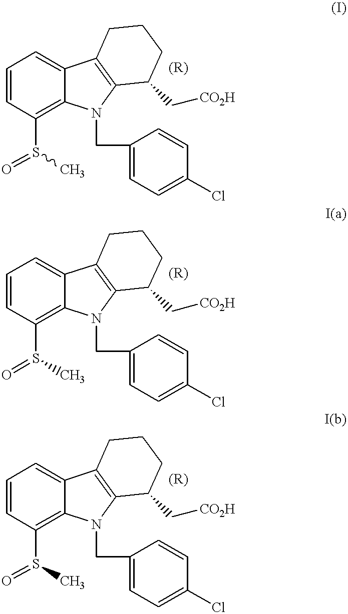

In one embodiment, the present invention provides substantially pure 2-[(1R)-9-(4-chlorobenzyl)-8-((S)-methylsulfinyl)-2,3,4,9-tetrahydro-1H-carbazol-1-yl]acetic acid I(a) and pharmaceutically acceptable salts thereof.

In another embodiment, the present invention provides substantially pure 2-[(1R)-9-(4-chlorobenzyl)-8-((R)-methylsulfinyl)-2,3,4,9-tetrahydro-1H-carbazol-1-yl]acetic acid I(b) and pharmaceutically acceptable salts thereof.

In another aspect, the present invention provides a pharmaceutical composition which comprises a compound of formula I, and a pharmaceutically acceptable carrier. As used hereinafter “formula I” is intended to include diastereomeric 1, as well as the individual diastereomers I(a) and I(b).

In yet another aspect, the present invention provides a method for the treatment of allergic conditions which comprises administering to a mammal an effective amount of a compound of formula I.

In yet another aspect, the present invention provides a method for preventing the action of prostaglandin D2 in a mammal which comprises administering to said mammal an effective amount of a compound of formula I.

As used herein, the following terms have the indicated meanings:

The term “substantially pure” means the the indicated stereoisomer is present in at least 95% by weight.

The term “treatment” includes alleviating, ameliorating, relieving or otherwise reducing, as well as preventing onset of symptoms commonly associated with allergic conditions and other prostaglandin D2 mediated diseases and disorders.

The term “effective amount” means that amount of the therapeutically active compound which provides a therapeutic benefit in the treatment, management, or prevention of allergic conditions or other prostaglandin D2 mediated diseases and disorders.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of formula I and pharmaceutically acceptable excipients.

While the diastereomeric mixture containing compounds I(a) and I(b) and their respective enantiomers is disclosed in U.S. Pat. No. 4,808,608, Applicants have now discovered that compounds I(a) and I(b), the 1R isomers, have much higher affinity for the prostaglandin D2 receptor than the other two diastereomers, which have limited affinity for the same receptor.

### Salts

Compounds of formula I have an acidic group as well as a basic nitrogen, and may form pharmaceutically acceptable salts with acids or bases. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, Iysine, methylglucamine, morpholine, piperazine, piperidine, polyamine resins, procaine, purines, theobromine, triethylamine, trimethylamine, tripropylamine, tromethamine, and the like.

Salts may also be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of formula I are meant to also include the pharmaceutically acceptable salts.

### Utilities

Compounds of formula I are potent and selective antagonists of the prostaglandin D2 receptor. The ability of these to antagonize the actions of prostaglandin D2 makes them useful for preventing, treating or reversing the symptoms, disorders or diseases induced by the binding of prostaglandin D2 to its receptors. Accordingly, another aspect of the present invention provides a method for the treatment (including prevention, alleviation, amelioration or suppression) of diseases or disorders or symptoms mediated by prostaglandin D2, which comprises administering to a mammal an effective amount of a compound of formula I. Such diseases, disorders, conditions or symptoms are for example: (1) allergic rhinitis (seasonal or perennial), (2) asthma, (3) allergic conjunctivitis, (4) urticaria or atopic dermatitis, and (5) nasal congestion.

### Dose Ranges

The magnitude of prophylactic or therapeutic dose of a compound of formula I will, of course, vary with the nature of the severity of the condition to be treated and with the particular compound of formula I and its route of administration. It will also vary according to the age, weight and response of the individual patient. In general, the daily dose range lies within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.001 mg to about 25 mg (preferably from 0.01 mg to about 1 mg) of a compound of formula I per kg of body weight per day and for cytoprotective use from about 0.1 mg to about 100 mg (preferably from about 1 mg to about 100 mg and more preferably from about 1 mg to about 10 mg) of a compound of formula I per kg of body weight per day.

In the case where an oral composition is employed, a suitable dosage range is, e.g. from about 0.01 mg to about 100 mg of a compound of formula I per kg of body weight per day, preferably from about 0.1 mg to about 10 mg per kg of body weight per day.

For the treatment of diseases of the eye, ophthalmic preparations for ocular administration comprising 0.001-1% by weight solutions or suspensions of the compounds of formula I in an acceptable ophthalmic formulation may be used.

### Pharmaceutical Compositions

Another aspect of the present invention provides pharmaceutical compositions which comprises a compound of formula I and a pharmaceutically acceptable carrier. Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of formula I in suitable propellants, such as fluorocarbons or hydrocarbons and dry powder inhalation (DPI) aerosol, which may be formulated as a dry powder of a compound of formula I with or without additional excipients.

Suitable topical formulations of a compound of formula I include transdermal devices, aerosols, creams, ointments, lotions, dusting powders, and the like.

In practical use, the compounds of formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form, in which case solid pharmaceutical carriers are employed. If desired, tablets may be coated by standard aqueous or nonaqueous techniques.

In addition to the common dosage forms set out above, the compounds of formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

The following are examples of representative pharmaceutical dosage forms for the compounds of formula I:

| Injectable Suspension (I.M.) | mg/mL |
|---|---|
| Compound of formula I | 10 |
| Methylcellulose | 5.0 |
| Tween 80 | 0.5 |
| Benzyl alcohol | 9.0 |
| Benzalkonium chloride | 1.0 |
| Water for injection to a total | |
| volume of 1 mL | |
| Tablet | mg/tablet |
| Compound of formula I | 25 |
| Microcrystalline Cellulose | 415 |
| Povidone | 14.0 |
| Pregelatinized Starch | 43.5 |
| Magnesium Stearate | 2.5 |
| | 500 |
| Capsule | mg/capsule |
| Compound of formula I | 25 |
| Lactose Powder | 573.5 |
| Magnesium Stearate | 1.5 |
| | 600 |
| Aerosol | Per canister |
| Compound of formula I | 24 mg |
| Lecithin, NF Liq. Conc. | 1.2 mg |
| Trichlorofluoromethane, NF | 4.025 g |
| Dichlorodifluoromethane, NF | 12.15 g |

### Combination Therapy

Compounds of formula I may be used in combination with other drugs that are used in the treatment/prevention/suppression or amelioration of the diseases or conditions for which compounds of formula I are useful. Such other drugs may be administered, by a route and in an amount commonly used therefor, contemporaneously or sequentially with a compound of formula I. When a compound of formula I is used contemporaneously with one or more other drugs, a pharmaceutical composition containing such other drugs in addition to the compound of formula I is preferred. Accordingly, the pharmaceutical compositions of the present invention include those that also contain one or more other active ingredients, in addition to a compound of formula I. Examples of other active ingredients that may be combined with a compound of formula I, either administered separately or in the same pharmaceutical compositions, include, but are not limited to: (a) VLA-4 antagonists such as those described in U.S. Pat. No. 5,510,332, WO97/03094, WO97/02289, WO96/40781, WO96/22966, WO96/20216, WO96/01644, WO96/06108, WO95/15973 and WO96/31206; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib; (h) inhibitors of phosphodiesterase type IV (PDE-IV); (i) antagonists of the chemokine receptors, especially CCR-1, CCR-2, and CCR-3; (j) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (metformin), a-glucosidase inhibitors (acarbose) and glitazones (troglitazone, pioglitazone, englitazone, MCC-555, BRL49653 and the like); (1) preparations of interferon beta (interferon beta-1a, interferon beta-1b); (m) anticholinergic agents such as muscarinic antagonists (ipratropium bromide); (n) other compounds such as 5-aminosalicylic acid and prodrugs thereof, antimetabolites such as azathioprine and 6-mercaptopurine, and cytotoxic cancer chemotherapeutic agents.

The weight ratio of the compound of the formula I to the second active ingredient may be varied and will depend upon the effective dose of each ingredient. Generally, an effective dose of each will be used. Thus, for example, when a compound of the formula I is combined with an antihistamine the weight ratio of the compound of the formula I to the antihistamine will generally range from about 1000:1 to about 1:1000, preferably about 200:1 to about 1:200. Combinations of a compound of the formula I and other active ingredients will generally also be within the aforementioned range, but in each case, an effective dose of each active ingredient should be used.

Compounds of the present invention may be prepared according to the reaction steps shown in Schemes 1 and 2.

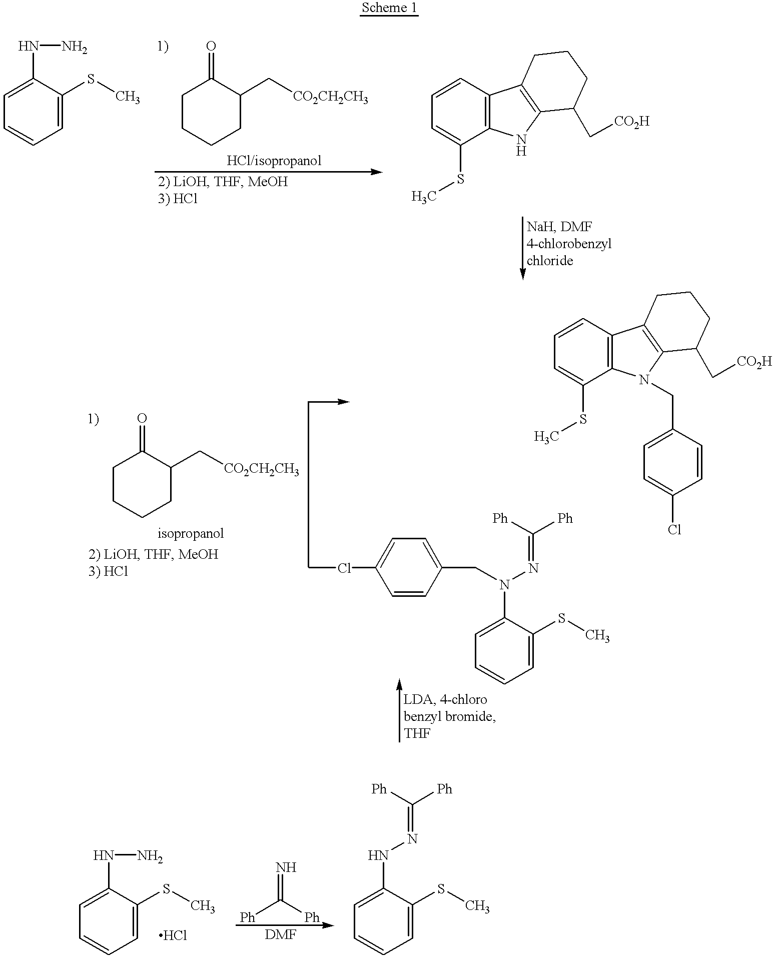

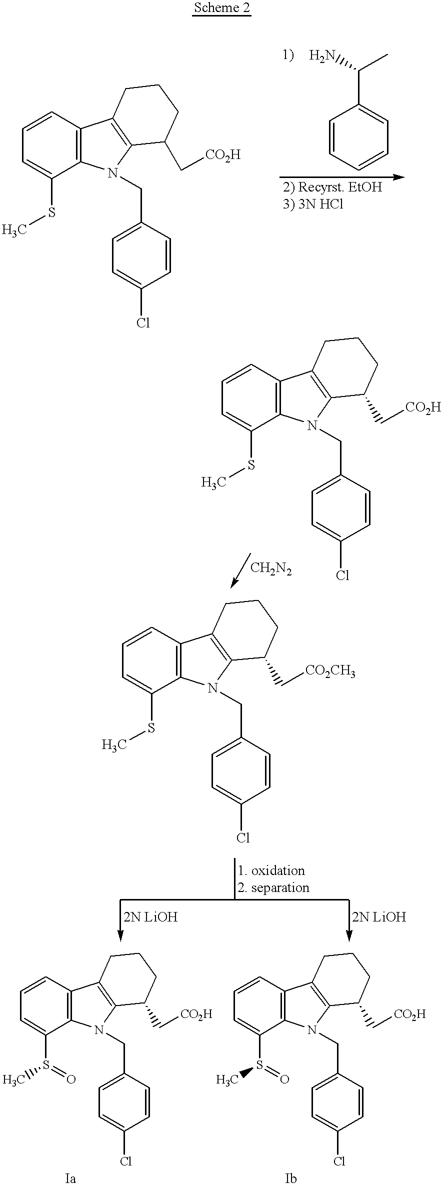

Scheme I depicts two synthetic routes for the preparation of 2-[9-(4-chlorobenzyl)-8-methylsulfanyl)-2,3,4,9-tetrahydro-1H-carbazol-1-yl]acetic acid, as a racemic mixture, from 1-[2-(methylsulfanyl)phenyl]hydrazine. In the first 1-[2-(methylsulfanyl)phenyl]hydrazine is reacted with ethyl 2-oxocyclohexaneacetate in isopropyl alcohol and in the presence of HCl to provide ethyl 2-[8-(methylsulfanyl)-2,3,4,9-tetrahydro-1H-carbazol-1-yl]acetate, which upon saponification provides the corresponding tetrahydrocarbazoleacetic acid. The tetrahydrocarbazoleacetic acid is treated with a base such as sodium hydride, followed by 4-chlorobenzyl chloride to provide the desired 2-[9-(4-chlorobenzyl)-8-methylsulfanyl)-2,3,4,9-tetrahydro-1-H-carbazol-1-yl]acetic acid.

Alternatively, 1-[2-(methylsulfanyl)phenyl]hydrazine is treated with benzophenone imine to provide the corresponding hydrazone adduct. The p-chlorobenzyl moiety is introduced by treating the hydrazone with a base such as lithium diisopropylamide followed by 4-chlorobenzyl bromide to provide diphenylmethanone N-(4-chlorobenzyl)-N-[2-(methylsulfanyl)phenyl]hydrazone. The desired tetrahydrocarbazoleacetic acid product is obtained by treating the hydrazone with ethyl 2-oxocyclohexaneacetate, followed by saponification.

In Scheme 2, the racemic mixture of 2-[9-(4-chlorobenzyl)-8-methylsulfanyl)-2,3,4,9-tetrahydro-1H-carbazol-1-yl]acetic acid is resolved using (R)-(+)-1-(1-naphthyl)ethylamine to provide the desired enantiomer, which is then converted to its methyl ester using diazomethane. Oxidation using, for example, m-chloroperbenzoic acid gives the corresponding sulfoxide as a mixture of two diastereomers, which are separated using conventional technique, such as high pressure liquid chromatography. Alternatively, chiral oxidation reagents such as (3′S, 2R) N-phenylsulfonyl 3,3-dichlorocamphoryl oxaziridine, give mixtures enriched in one or the other epimer at the sulfur stereogenic center. The two diastereomers are separately treated with a base to generate their corresponding acids.

As prostaglandin D2 antagonistic activity is mostly attributable to the 1R isomers (formula I), it may not be necessary to separate the two diastereomers resulting from the oxidation, and the mixture of diastereomers may be directly hydrolyzed to provide the corresponding mixture of acids.

The following examples are provided to illustrate the invention, and are not to be construed as limiting the scope thereof in any manner.

### EXAMPLE 1

Preparation of 2-[(1R)-9-(4-chlorobenzyl)-8-((R)-methylsulfinyl)-2,3,4,9-tetrahydro-1H-carbazol-1-yl]acetic acid (Ib) and 2-[(1R)-9-(4-chlorobenzyl)-8-((S)-methylsulfinyl)-2,3,4,9-tetrahydro-1H-carbazol-1-yl]acetic acid (Ia)

### Step a(1). 1-[2-(methylsulfanyl)phenyl]hydrazine

2-(Methylthio)aniline (30 g, 215 mmol) was dissolved in 2N HCl (215 ml) and cooled to 0° C. and a solution of NaNO₂ (16.3 g, 237 mmol) in 50 ml water was added dropwise (maintaining the temperature below 5° C). After 10 min the solution was added portionwise to a solution of Na₂S₂O₄ (220 g 85% pure, 1075 mmol) in a biphasic mixture of 1200 ml of ether and 1200 mL of water dropwise (maintaining the temperature below 5° C). After stirring for one hour at 0° C. the mixture was warmed to room temperature and the pH set to 10 with 2N NaOH. The ether layer was separated and the aqueous layer washed once with ether. The combined organic layers were dried with sodium sulfate, the solvent removed and the product purified on silica with 25% ethyl acetate/hexane to provide 15.7 g of the title compound (47%).

¹H NMR (400 MHz), DMSO, δ: 2.30 (s, 3H); 4.10 (s, 2H); 6.20 (s, 1H); 6.60 (t, 1H); 7.10 (m, 2H); 7.20 (d, 2H).

### Step a(2). 1-[2-(methylsulfanyl)phenyl]hydrazine hydrochloride

Bromothioanisole (414 g, 2041 mmol) was added dropwise to a suspension of Mg (54.6 g, 2245 mmol) in 1000 ml tetrahydrofuran under N₂ (maintaining a gentle reflux). The mixture was refluxed for 2 hours and cooled to −78° C. Solid di-tert-butyl azodicarboxylate (470 g, 2041 mmol) was added portionwise maintaining the temperature below −50° C. The mixture was stirred for 10 min, warmed to −30° C. and quenched with 1 eq of acetic acid, 1000 ml of water and 1000 mL of ether. After agitation the ether layer was collected and dried with sodium sulfate. The solvent was removed and the crude di(tert-butyl) 1-[2-(methylsulfanyl)-phenyl]-1,2-hydrazinedicarboxylate used as is in the next step.

Crude di(tert-butyl) 1-[2-(methylsulfanyl)phenyl]-1,2-hydrazinedicarboxylate was dissolved in 8000 ml of 1M HCl in ether. HCl gas was bubbled through the mixture for approximately 10 min every 2 hours, over a period of 6 hours. The mixture was stirred overnight and a precipitate formed. The solid was collected by filtration and washed with ether to provide 262 g of the title compound (69% from bromothioanisole).

¹H NMR (400 MHz), DMSO, δ: 2.40 (s, 3H); 7.00 (m, 2H); 7.20 (t, 1H); 7.35 (d, 1H); 7.70 (s, 1H); 10.15 (s, 3H).

### Step b. ethyl 2-[8-(methylsulfanyl)-2,3,4,9-tetrahydro-1H-carbazol-1-yl]acetate

Method A. 1-[2-(methylsulfanyl)phenyl]hydrazine (15.7 g, 102 mmol) and ethyl 2-cyclohexanoneacetate (18.7 g, 102 mmol) were dissolved in 300 ml isopropanol containing 1 eq HCl. The mixture was refluxed overnight under nitrogen then cooled to room temperature. The solvent was stripped and the residue partitioned between 300 ml of water and 300 mL of dichloromethane. The water layer was washed with dichloromethane, and the organic layers were combined, dried with sodium sulfate and the solvent removed. The mixture was purified on silica with 5% ethyl acetate/toluene to provide 14.2 g (46%) of the title compound.

Method B. 1-[2-(methylsulfanyl)phenyl]hydrazine hydrochloride (50 g, 262 mmol) and ethyl 2-cyclohexanoneacetate (48.3 g, 262 mmol) were dissolved in 1300 ml isopropanol. The mixture was refluxed overnight under nitrogen then cooled to room temperature. The solvent was stripped and the residue partitioned between 1300 ml water and ethyl acetate. The water layer was washed with ethyl acetate, and the organic layers were combined, dried with sodium sulfate and the solvent removed. The mixture was purified on silica with 2.5% ethyl acetate/toluene to provide 42 g crude title compound.

¹H NMR (400 MHz), DMSO, δ: 1.20 (t, 3H); 1.60 (m, 1H); 1.70 (m, 1H); 1.80 (m, 1H); 1.95 (m, 1H); 2.30-2.45 (m, 1H); 2.45 (s, 3H); 2.55 (t, 2H); 3.20 (dd, 1H); 3.30 (m, 1H); 4.10 (q, 2H); 6.90 (t, 1H); 7.00 (d, 1H); 7.25 (d, 1H); 10.60 (s, 1H).

### Step c. 2-[8-(methylsulfanyl)-2,3,4,9-tetrahydro-1H-carbazol-1-yl]acetic acid (5)

Crude product of step b (42 g) was dissolved in 400 ml of tetrahydrofuran and methanol 1:1 and 207 ml of 2N LiOH was added thereto. The mixture was refluxed for 30 min and cooled to room temperature. The organic solvents were removed and 800 ml of 1N HCl and 800 mL of ethyl acetate added. The layers were separated and the aqueous layer washed with ethyl acetate. The combined organic layers were dried with sodium sulfate and the solvent removed. The resulting solid was triturated with 200 ml 5% ether/hexane to provide 30.4 g of the title compound.

¹H NMR (400 MHz), DMSO, δ: 1.60 (m, 1H); 1.70 (m, 1H); 1.80 (m, 1H); 1.95 (m, 1H); 2.30 (q, 1H); 2.45 (s, 3H); 2.55 (s (broad), 2H); 3.10 (dd, 1H); 3.25 (m, 1H); 6.90 (t, 1H); 7.00 (d, 1H); 7.25 (d, 1H); 10.55 (s, 1H); 12.25 (s, 1H).

### Step d. 2-[9-(4-chlorobenzyl)-8-(methylsulfanyl)-2,3,4,9-tetrahydro-1H-carbazol-1-yl]acetic acid

The product of step c in 100 ml dimethylformamide (30.4 g, 110 mmol) was added to a suspension of a 60% NaH dispersion in mineral oil (11 g, 276 mmol) in 500 ml dimethylformamide at −78° C. under N₂. The mixture was warmed to room temperature, stirred for 30 min and then cooled to −78° C. A solution of 220 mmol of 4-chlorobenzyl chloride in 100 ml of dimethylformamide was added thereto, and the mixture warmed to room temperature and stirred for 4 hours. 500 ml of 1N HCl and 500 mL of isopropyl acetate were added. The layers were separated and the organic layer washed 2 times with water. The organic layer was dried with sodium sulfate and the solvent removed. The resulting residue was purified on a plug of silica to provide 35 g of the title compound.

¹H NMR (400 MHz), DMSO, δ: 1.60-1.90 (m, 4H); 2.30 (s, 3H); 2.35-2.40 (m, 2H); 2.60 (m, 1H); 2.85 (m, 1H); 3.20 (d, 1H); 5.50 (d, 1H); 6.00 (d, 1H); 6.70 (d, 2H); 7.05 (m, 2H); 7.30 (m, 3H); 12.30 (s, 1H).

### Step e. 2-[(1R)-9-(4-chlorobenzyl)-8-(methylsulfanyl)-2,3,4,9-tetrahydro-1H-carbazol-1-yl]acetic acid

The racemic acid of step d (35 g, 91.3 mmol) was dissolved in dry ethanol (900 mL) and heated to reflux. (R)-(+)-1-(1-naphthyl)ethylamine (15.64 g, 91.3 mmol, 1 eq) was added and the reaction mixture was stirred at 80° C. for 30 min, then allowed to cool slowly to room temperature. The resulting suspension was stirred for 16 hours, and then the salt was filtered and air dried for 2 hours to yield 15.2 g of white solid. The solid was recrystallized in ethanol (700 mL) to afford 13.4 g of salt. It was suspended in methanol (200 mL) and acidified with 3N HCl (11.5 mL), and the resulting solution was concentrated to dryness and the residue was partitioned in 1:1 ethyl acetate/H₂O. Organic fraction was dried with Na₂SO₄, and concentrated to give 9.4 g of solid.

The acid was analyzed by HPLC on chiralpak AD (250×4.6 mm). Elution was performed with a mixture of 5% 2-propanol in hexane and 0.2% acetic acid. A retention time of 8.4 min. was observed and the acid was obtained in 99.7% ee.

### Step f. methyl 2-[(1R)-9-(4-chlorobenzyl)-8-(methylsulfanyl)-2,3,4,9-tetrahydro-1H-carbazol-1-yl]acetate

The acid of step e (8.0 g, 20.0 mmol) was dissolved in acetone (250 mL) and treated with diazomethane (approximately 2M solution in diethyl ether) until yellow color remained. Excess CH₂N₂ was quenched with acetic acid, and the reaction mixture was concentrated to dryness to afford a yellow oil (8.3 g). (100%).

H NMR (acetone d₆) δ7.37 (d, 1H), 7.26 (d, 2H), 7.15 (d, 1H), 7.03 (t, 1H), 6.78 (d, 2H), 6.2 (d, 1H), 5.65 (d, 1H), 3.65 (s, 3H), 3.4-3.3 (m, 1H), 2.81-2.75 (m, 1H), 2.66-2.5 (m, 3H), 2.3 (s, 3H), 1.93-1.75 (m, 4H).

### Step g. methyl 2-[(R)-9-(4-chlorobenzyl)-8-((S)-methylsulfinyl)-2,3,4,9-tetrahydro-1H-carbazol-1-yl]acetate Me-(Ib) and methyl 2-[(1R)-9-(4-chlorobenzyl)-8-((R)-methylsulfinyl)-2,3,4,9-tetrahydro-1H-carbazol-1-yl]acetate Me-(Ia)

The sulfide of step f (8.3 g, 20.0 mmol) was dissolved in dichloromethane (300 mL) and m-chloroperbenzoic acid (4.0 g @ 85%, 20.0 mmol, 1 eq) was added. The mixture was stirred at room temperature for 30 min, washed with saturated NaHCO₃ (2×25 mL), dried with sodium sulfate, and concentrated to dryness to give 8.6 g of yellow foam.

The product was a mixture of two diastereomers which was separated by HPLC on Zorbax Pro 10 process column, eluting with 25% 2-propanol in hexane to provide 3.46 g of the less polar diastereomer Me-(Ib) and 2.72 g of the more polar diastereomer were recovered Me-(Ia).

¹H NMR (acetone d6) Less polar compound: δ7.8 (d, 1H), 7.66 (d, 1H), 7.35-7.25 (m, 3H), 6.8 (d, 2H), 5.78 (d, 1H), 5.41 (d, 1H), 3.6 (s, 3H), 3.43-3.35 (m, 1H), 2.9-2.6 (m, 2H), 2.52 (d, 2H), 2.3 (s, 3H), 2.0-1.85 (m, 4H). More polar compound: δ7.77 (d, 1H), 7.65 (d, 1H), 7.35-7.25 (m, 3H), 6.75 (d, 2H), 5.58 (d, 1H), 5.42 (d, 1H), 3.65 (s, 3H), 3.4-3.3 (m, 1H), 2.9-2.56 (m, 4H), 2.54 (s, 3H), 2.0-1.85 (m, 4H).

### Step h(1). 2-[(1R)-9-(4-chlorobenzyl)-8-((R)-methylsulfinyl)-2,3,4,9-tetrahydro-1H-carbazol-1-yl]acetic acid (formula I(b))

The less polar ester of step g (2.36 g, 5.5 mmol) was dissolved in 25 mL of tetrahydrofuran:methanol (3:1 mixture) and 2N LiOH (7.1 mmol, 1.3 eq) was added. The reaction mixture was stirred at room temperature for 2 hours and a white suspension was obtained. When acidified to pH 2 with 1N HCl the reaction mixture became clear. After stirring at room temperature for 1 hr, the acid product precipitated. The solid was filtered and washed with small volume of ethyl acetate to afford 2.1 g (92%) of the title compound.

¹H NMR (DMSO d6): δ7.7 (d, 1H), 7.65 (d, 1H), 7.35 (d, 2H), 7.27 (t, 1H), 6.72 (d, 2H), 5.62 (d, 1H), 5.38 (d, 1H), 2.8 (d, 1H), 2.65-2.5 (m, 1H), 2.38-2.28 (m, 2H), 2.35 (s, 3H), 1.92-1.75 (m, 4H). Optical rotation: +121.3° (c=0.39 in methanol).

### Step h(2) 2-[(1R)-9-(4-chlorobenzyl)-8-((S)-methylsulfinyl)-2,3,4,9-tetrahydro-1H-carbazol-1-yl]acetic acid (formula I(a))

The more polar ester of step g (1.6 g, 3.7 mmol) was dissolved in 15 mL of tetrahydrofuran:methanol (3:1 mixture) and 2N LiOH (4.8 mmol, 1.3 eq) was added. The reaction mixture was stirred at room temperature for 2 hours and a white suspension was obtained. When acidified to pH 2 with 1N HCl the reaction mixture became clear. After stirring at room temperature for 1 hr, acid product precipitated. The solid was filtered and washed with small volume of ethyl acetate to afford 1.37 g (89%) of the title compound.

hu 1H NMR (DMSO d6): δ7.66 (d, 1H), 7.63 (d, 1H), 7.34 (d, 2H), 7.28 (t, 1H), 6.69 (d, 2H), 5.42 (d, 1H), 5.24 (d, 1H), 3.2 (d, 1H), 2.8 (d, 1H), 2.68-2.54 (m, 2H), 2.58 (s, 3H), 2.47-2.39 (m, 1H), 1.9-1.75 (m, 4H). Optical rotation: −231.9 (c=0.31 in methanol).

### EXAMPLE 2

The compound of Example 1, step d may also be prepared as follows:

### Step a. diphenylmethanone N-[2-(methylsulfanyl)phenyl]hydrazone

1-[2-(Methylsulfanyl)phenyl]hydrazine hydrochloride (30 g, 148 mmol) was dissolved in 300 ml dimethylformamide and benzophenone imine (26.7 g, 148 mmol) was added dropwise over 5 min. The mixture was stirred for 1 hour and 300 ml ether and 300 mL of water were added. The layers were separated and the organic layer washed twice with brine. The organic layer was dried with sodium sulfate and the solvent removed. The residue with triturated with hexane to obtain 38.5 g of title compound (containing 18 % benzophenone).

¹H NMR (400 MHz), DMSO, δ: 2.60 (s, 3H); 6.80 (t, 1H); 7.30-7.45 (m, 7H); 7.55 (d, 2H); 7.60 (t, 2H); 7.65 (s, 2H); 8.40 (s, 1H).

### Step b. diphenylmethanone N-(4-chlorobenzyl)-N-[2-(methylsulfanyl)phenyl]-hydrazone

Diisopropylamine (29 ml, 206 mmol) was dissolved in 50 ml tetrahydrofuran and cooled to 0° C. 76 ml n-BuLi (2M in c-Hexane) was added dropwise and the solution was stirred for 30 min. This solution was then cannulated into a solution of 61.6 g of the product of step a (containing 18% benzophenone) in 150 ml tetrahydrofuran at 0° C. The mixture was stirred at room temperature for 30 min, cooled to 0° C. and 4-bromobenzyl bromide (39.1 g, 190.3 mmol) in 50 ml tetrahydrofuran was added. The mixture was stirred for 30 min and 200 ml of NH₄Cl (sat) and ether were added. The layers were separated and the aqueous layer washed with ether. The combined organic layers were dried with sodium sulfate and the solvent removed. The residue was triturated with hexane to obtain 67 g of the title compound.

¹H NMR (400 MHz), DMSO, δ: 2.35 (s, 3H); 4.40 (s, 2H); 6.80-7.00 (m, 6H); 7.10 (t, 3H); 7.30 (m, 5H); 7.40 (d, 2H); 7.50 (d, 2H).

### Step c. 2-[9-(4-chlorobenzyl)-8-(methylsulfanyl)-2,3,4,9-tetrahydro-1H-carbazol-1-yl]acetic acid

The product of step b (84.6 g, 191 mmol) and ethyl 2-cyclohexanoneacetate (35.2 g, 191 mmol) were dissolved in 850 ml ethanol and p-toluenesulfonic acid (72.8 g, 381 mmol) was added. The mixture was refluxed for 3 hours, cooled to room temperature and the solvent stripped. 1000 ml ether and 1000 mL of water were added. The layers were separated and the organic layer washed with brine, dried with sodium sulfate and the solvent removed. The residue was purified on silica with 3% ethyl acetate/Hex. The crude product (43.6 g) contained 12% benzophenone and 22% of ethyl 2-[9-(4-chlorobenzyl)-2,3,4,9-tetrahydro-1H-carbazol-1-yl]acetate. 43.4 g of the crude mixture was dissolved in 500 ml of tetrahydrofuran and MeOH and 152 ml of 2N LiOH was added. The mixture was refluxed for 30 min and cooled to room temperature. The organic solvents were removed and 800 ml of 1N HCl and ethyl acetate added. The layers were separated and the aqueous layer washed with ethyl acetate. The combined organic layers were dried with sodium sufate and the solvent removed. The resulting solid was purified on a short silica column with 25% ethyl acetate/toluene/ 1% acetic acid to provide 32 g of the title compound, contaminated with ethyl 2-[9-(4-chlorobenzyl)-2,3,4,9-tetrahydro-1H-carbazol-1-yl]acetate.

¹H NMR (400 MHz), DMSO, δ: 1.60-1.90 (m, 4H); 2.30 (s, 3H); 2.35-2.40 (m, 2H); 2.60 (m, 1H); 2.85 (m, 1H); 3.20 (d, 1H); 5.50 (d, 1H); 6.00 (d, 1H); 6.70 (d, 2H); 7.05 (m, 2H); 7.30 (m, 3H); 12.30 (s, 1H).

### EXAMPLE 3

Alternate oxidation of methyl 2-[(1R)-9-(4-chlorobenzyl)-8-(methylsulfanyl)-2,3,4,9-tetrahydro-1H-carbazol-1-yl]acetate (product of step f, Example 1) to provide methyl 2-[(1R)-9-(4-chlorobenzyl)-8-((S)-methylsulfinyl)-2,3,4,9-tetrahydro-1H-carbazol-1-yl]acetate (Me-Ia) and methyl 2-[(1R)-9-(4-chlorobenzyl)-8-((R)-methylsulfinyl)-2,3,4,9-tetrahydro-1H-carbazol-1-yl]acetate (Me-Ib)

To a CCl₄ solution (5.0 mL) of methyl 2-[(1R)-9-(4-chlorobenzyl)-8-(methylsulfanyl)-2,3,4,9-tetrahydro-1H-carbazol-1-yl]acetate (40.0 mg, 0.1 mmol) at room temperature was added (−) (3′S, 2R) N-phenylsulfonyl 3,3-dichlorocamphoryl oxaziridine (27.5 mg, 0.08 mmol) and this was allowed to stir at room temperature overnight. At this time, the reaction was concentrated and the residue was purified by flash chromatography eluting with 70% EtOAc/hexane to provide 26.7 mg (86%) of the title sulfoxides in an 12:1 mixture of diastereomers as a clear colorless oil. The major diastereoisomer obtained by this procedure was separated by column chromatography as in example 2, and was identical to the previously isolated compound Ia.

### EXAMPLE 4

Radioligand binding assays using membranes from cells that express recombinant prostanoid DP receptor (DP).

Radioligand binding assays are conducted essentially as previously described (Abramovitz et al., Biochem. Biophys. Acta 1483-2, 285-293, 2000). HEK293(EBNA) cells expressing DP are grown in supplemented DMEM complete medium at 37° C. in a humidified atmosphere of 6% CO₂ in air, and then harvested. Cells are disrupted by nitrogen cavitation at 800 psi for 30 min. on ice in the presence of protease inhibitors (2 mM phenylmethylsulfonylfluoride, 10 μM E-64, 100 μM leupeptin and 0.05 mg/mL pepstatin). Membranes are prepared by differential centrifugation (1000×g for 10 min, then 160,000×g for 30 min, all at 4° C.). The 160,000×g pellets are resuspended in 10 mM HEPES/KOH (pH 7.4) containing 1 mM EDTA at approximately 5-10 mg/mL protein by Dounce homogenization (Dounce A; 10 strokes), frozen in liquid nitrogen and stored at −80° C. DP receptor binding assays are performed in a final incubation volume of 0.2 mL in 10 mM HEPES/KOH (pH 7.4), containing 1 mM EDTA, 10 mM MnCl₂, 0.7 nM [³H]PGD₂ (115-200 Ci/mmol). The reaction was initiated by addition of 30-60 μg membrane protein from the 160,000×g fraction. Test compounds are added in dimethylsulfoxide (Me₂SO) at 1% (v/v) in all incubations. Non-specific binding was determined in the presence of 1-10 μM of non-radioactive PGD₂. Incubations are conducted for 60 min. at room temperature. Incubations are terminated by rapid filtration at 4° C. Radioactivity bound to the individual filters is determined by scintillation counting. Maximum specific binding is defined as the total binding minus the non-specific binding. Specific binding is determined at each concentration of test compound and is expressed as a percentage of the maximum specific binding. Sigmoidal equilibrium competition curves were constructed by expressing percentage maximum specific binding as a function of ligand concentration and analyzed by a validated custom designed software employing a simplex driven non-linear least-squares curve fitting routine based on a four parameter equation to determine the inflection point concentration (InPt). The corresponding Kᵢvalues were derived from the equation Kᵢ=InPt/1+([radioligand]/K_{D}). The affinities of compounds Ia, Ib and their respective enantiomers for the prostaglandin D receptor are shown in Table 1.

TABLE 1
Affinity (Ki) for the prostaglandin D receptor of Ia, Ib
and their enantiomers

| | | |
|---|---|---|
| Compound Ia | 1.7 ± 0.3 nM | n = 5 |
| Compound Ib | 2.1 ± 0.1 nM | n = 5 |
| Enantiomer of Ia | 119 ± 39 nM | n = 5 |
| Enantiomer of Ib | 72 ± 4 nM | n = 5 |

What is claimed is:

1. Substantially pure 2-[( 1R)-9-(4-chlorobenzyl)-8-((R)-methyl-sulfinyl)-2,3,4,9-tetrahydro-1H-carbazol-1-yl]acetic acid or a pharmaceutically acceptable salt thereof.

2. Substantially pure 2-[(1R)-9-(4-chlorobenzyl)-8-((S)-methyl-sulfinyl)-2,3,4,9-tetrahydro-1H-carbazol-1-yl]acetic acid of a pharmaceutically acceptable salt thereof.

3. A pharmaceutical composition comprising the compound of claim 1 and a pharmaceutically acceptable carrier.

4. A pharmaceutical composition comprising the compound of claim 2 and a pharmaceutically acceptable carrier.

5. A method for the treatment of allergic conditions in a mammal comprising administering to said mammal an effective amount of the compound of claim 1.

6. A method for the treatment of allergic conditions in a mammal comprising administering to said mammal an effective amount of the compound of claim 2.

7. Substantially pure ²-[(1R)-⁹-(4-chlorobenzyl)-8-methyl-sulfinyl-2,3,4,9-tetrahydro-1H-carbazol-1-yl]acetic acid consisting essentially of 2-[(1R)-9-(4-chlorobenzyl)-8-((S)-methylsulfinyl)-2,3 ,4,9-tetrahydro-1H-carbazol-1-yl]acetic acid and 2-[(1R)-9-(4-chlorobenzyl)-8-((R)-methylsulfinyl)-2,3,4,9-tetrahydro-1H-carbazol1-yl]acetic acid, and pharmaceutically acceptable salts thereof.

8. A pharmaceutical composition comprising the compound of claim 7 and a pharmaceutically acceptable carrier.

9. A method for the treatment of allergic conditions in a mammal comprising administering to said mammal an effective amount of the compound of claim 7.

10. A method for the treatment of allergic rhinitis in a human comprising administering to said human an effective amount of the compound of claim 1.

11. A method for the treatment of allergic rhinitis in a human comprising administering to said human an effective amount of the compound of claim 2.

12. A method for the treatment of allergic rhinitis in a human comprising administering to said human an effective amount of the compound of claim 7.

13. A method for the treatment of allergic rhinitis and the relief of nasal congestion in a human comprising administering to said human an effective amount of the compound of claim 1.

14. A method for the treatment of allergic rhinitis and the relief of nasal congestion in a human comprising administering to said human an effective amount of the compound of claim 2.

15. A method for the treatment of allergic rhinitis and the relief of nasal congestion in a human comprising administering to said human an effective amount of the compound of claim 7.

16. A method for the treatment of diseases mediated by prostaglandin D2 in a human comprising administering to said human an effective amount of the compound of claim 1.

17. A method for the treatment of diseases mediated by prostaglandin D2 in a human comprising administering to said human an effective amount of the compound of claim 2.

18. A method for the treatment of diseases mediated by prostaglandin D2 in a human comprising administering to said human an effective amount of the compound of claim 7.
